(19) United States
(12) Patent Application Publication
(10) Pub. No.: US 2026/0174927 A1
(43) Pub. Date: Jun. 25, 2026

(54) DRESSING FORMULATIONS

(71) Applicant: Hemostasis, LLC, St. Paul, MN (US)

(72) Inventors: John H. BURBAN, Lake Elmo, MN (US); John W. SHANAHAN, White Bear Lake, MN (US); Michael R. SPEARMAN, Woodlands, TX (US); Keith A. ROBERTS, Dellwood, MN (US); Peter J. CATALANO, Watertown, MA (US)

(21) Appl. No.: 19/128,231

(22) PCT Filed: Nov. 8, 2023
(86) PCT No.: PCT/US2023/036999

§ 371 Date: May 8, 2025

Related U.S. Application Data
(60) Provisional application No. 63/424,266, filed on Nov. 10, 2022.

(51) Int. Cl. A61L 15/64; A61L 15/28; A61L 15/44

(52) U.S. Cl. CPC A61L 15/64; A61L 15/28; A61L 15/44; A61L 2300/222; A61L 2300/442

(57) ABSTRACT

In one aspect, the invention provides a dressing comprising a matrix with a first ionic state and an agent with a second ionic state, said agent having an activity selected from the group consisting of a bactericidal activity, a fungicidal activity, and an antiviral activity, wherein said first ionic state is different from said second ionic state. In some embodiments, the dressing further comprises a steroid. In another aspect, the invention provides dry porous compound comprising a matrix with a first ionic state and an agent with a second ionic state, said agent having an activity selected from the group consisting of a bactericidal activity, a fungicidal activity, and an antiviral activity, said first ionic state being different from said second ionic state, wherein upon hydration of the compound with an expansion liquid, the hydrated compound expands to form the dressing described herein.

### CROSS REFERENCE TO RELATED APPLICATION

This application claims priority benefit from U.S. Provisional Application No. 63/424,266, filed Nov. 10, 2022, the entire contents of which is hereby incorporated by reference.

### BACKGROUND

The invention relates to the field of medicine and particularly anti-microbial wound dressings.

It is well known that to promote wound healing, the wound must be kept clean and preferably free from infection. The invention of antibiotics, such as penicillin, have improved wound care and treatment. However, with the evolution of antibiotic-resistant bacteria, and with the growing number of viruses and fungi that have affected human patients, the development of dressing and formulations that are bactericidal, fungicidal, and/or antiviral that aid wound healing is needed.

### SUMMARY

The invention provides an antimicrobial formulation that, when applied as a dressing next to a healing wound, can slowly elute an antimicrobial agent while at the same time degrading such that the dressing need never be removed when the wound is fully healed.

Accordingly, in a first aspect, the invention provides three-dimensional dressing comprising a matrix with a first ionic state and an agent with a second ionic state, said agent having an activity selected from the group consisting of a bactericidal activity, a fungicidal activity, and an antiviral activity, wherein said first ionic state is different from said second ionic state. In some embodiments, the agent has antimicrobial activity.

In some embodiments, the first ionic state is an anionic state, and the second ionic state is a cationic state. In some embodiments, the first ionic state is a cationic state and the second ionic state is an anionic state.

In some embodiments, the matrix is a polymer of chitosan. In some embodiments, the matrix is a polymer of N,O-carboxymethyl chitosan (NOCC). In some embodiments, wherein the agent is selected from the group consisting of methyl blue and gentian violet.

In some embodiments, the dressing further comprises a second matrix with a nonionic state. In some embodiments, the dressing further comprises a second matrix with a cationic state. In some embodiments, the second matrix is hydroxyethyl cellulose.

In some embodiments, the agent enhances a structural property of the dressing as compared to the matrix alone. In some embodiments the dressing can be used as an anti-adhesion barrier, spacer such as but not limited to an intranasal splint, or a hemostat dressing. In some embodiments, the dressing can be used a nasal hemostat to treat epistaxis. In some embodiments, elution of the agent from the dressing is achieved by contacting the dressing with an elution liquid. In some embodiments, elution of the agent from the dressing degrades the dressing. In some embodiments, the dressing is degraded when placed into a lumen of a body cavity adjacent to a wall of said lumen of said cavity, wherein said cavity produces moisture or is able to receive moisture, said moisture acting as an elution liquid when in contact with the dressing. In some embodiments, wherein the elution liquid is water. In some embodiments, wherein the elution liquid contains a physiologically normal amount of sodium chloride. In some embodiments, the elution liquid is physiologically normal saline. In some embodiments, the moisture is from mucous. In some embodiments the elution liquid contains a medication such as a steroid. In some embodiments, the mucous is produced by the wall of the body cavity. In some embodiments, the body cavity is a nasal cavity.

In some embodiments, the speed of elution of the agent from the dressing increases by increasing ionic strength of the elution liquid.

In some embodiments, the dressing can be used in patients undergoing nasal/sinus surgery as a space occupying hemostat or splint to, for example, separate tissue or structures compromised by surgical trauma; and/or separate and prevent adhesions between mucosal surfaces during mesothelial cell regeneration in the nasal cavity; and/or help control minimal bleeding following surgery or trauma; and/or help control minimal bleeding following surgery or nasal trauma by tamponade effect, blood absorption and platelet aggregation; and/or act as an adjunct to aid in the natural healing process.

In some embodiments, the dressing has a shape with a length, width, and thickness and the agent elutes from the dressing into the wall of the lumen of the body cavity a distance of not more than 50% of the longest of said length, width, or thickness. It shall be understood that the shape may be any shape including, for example, a circle, rectangle, square, trapezoid, or a custom shape designed to fit into the desired body cavity. In some embodiments, the dressing has a length, width, and thickness and the agent elutes from the dressing into the wall of the lumen of the body cavity a distance of not more than 25% of the longest of said length, width, or thickness. In some embodiments, the dressing has a length, width, and thickness and the agent elutes from the dressing into the wall of the lumen of the body cavity a distance of not more than 10% of the longest of said length, width, or thickness.

In some embodiments, wherein the dressing serves as an adhesion barrier to cells aligning (or forming) the wall of the body cavity. In some embodiments, cells in the wall of the lumen of the body cavity will not adhere directly to the dressing. In some embodiments, the dressing prevents cells in the wall of the lumen of the body cavity from growing into the lumen and reducing the diameter of the lumen.

In some embodiments, wherein dressing adheres to ionic compounds in the mucous. In some embodiments, wherein the ionic compounds have a positive charge.

In some embodiments, the dressing consists essentially of a matrix consisting essentially of N,O-carboxymethyl chitosan (NOCC) and hydroxyethyl cellulose and an agent selected from the group consisting of gentian violet and methylene blue. In some embodiments, the dressing consists essentially of N,O-carboxymethyl chitosan (NOCC), hydroxyethyl cellulose, gentian violet, and methylene blue. In some embodiments, the dressing consists essentially of N,O-carboxymethyl chitosan (NOCC) and an agent selected from the group consisting of gentian violet and methylene blue. In some embodiments, the dressing consists essentially of N,O-carboxymethyl chitosan (NOCC), gentian violet, and methylene blue. In some embodiments, the dressing is free of a silver salt. In some embodiments, the dressing is free of a silver salt of a polymer. In some embodiments, the dressing is free of a silver salt of an anionic polycarboxylate polymer.

In some embodiments, the dressing further comprises a steroid. In some embodiments, the steroid is covalently bound to the matrix.

In another aspect, the invention provides a dry porous compound comprising a matrix with a first ionic state and an agent with a second ionic state, said agent having an activity selected from the group consisting of a bactericidal activity, a fungicidal activity, and an antiviral activity, said first ionic state being different from said second ionic state, wherein upon hydration of the compound with a liquid (e.g., an expansion liquid), the hydrated compound expands to form the three-dimensional dressing as described herein. The dry porous compound may be compressed to aid in the placement of the dressing.

In some embodiments, the dressing is a sponge. In some embodiments, the dressing has a predetermined shape. In some embodiments, the predetermined shape is designed to fit into a nasal passage of a human. In some embodiments, the compound is a relatively flat oval shape that, upon hydration, expands in one dimension. In some embodiments, the compound, upon hydration, expands in two dimensions (e.g., expands laterally and longitudinally). In some embodiments, the compound expands in three dimensions (e.g., expands laterally, longitudinally, and vertically). In some embodiments, the expansion is at least four-fold and less than ten-fold. In some embodiments, the expansion is at about five-fold. In some embodiments, the expansion is at least about six-fold. In some embodiments, the expansion is about seven-fold.

In some embodiments, wherein the expansion liquid is water. In some embodiments, wherein the expansion liquid contains a physiologically normal amount of sodium chloride. In some embodiments, the expansion liquid is physiologically normal saline.

In various embodiments, the elution liquid and the expansion liquid are the same. In various embodiments, the elution liquid and the expansion liquid are different.

### BRIEF DESCRIPTION OF THE DRAWINGS

FIGS. 1A-1C are photographs of a non-limiting example of a dressing of the invention in its compressed form.

FIGS. 2A-2C are photographs of a non-limiting example of a dressing of the invention in its expanded form following hydration of the compressed dressing with expansion liquid.

### DETAILED DESCRIPTION

All the patents and scientific publications cited herein are herein incorporated by reference in their entireties to the same extent as if each was specifically stated as being entirely incorporated by reference herein. Any conflict between any reference cited herein and the specific teachings of this specification shall be resolved in favor of the latter.

U.S. Pat. Nos. 5,811,471; 6,183,764; 6,361,786; 5,709,672; 7,993,670; 8,227,656; 8,124,826; 8,962,908; and 10,143,771 describe various dressings and other medical devices.

In some embodiments, the invention provides a dressing for use following sinus surgery to prevent cell adhesion to the walls of the nasal cavity, control mild bleeding and provide a level of antimicrobial effectiveness. In some embodiments, the dressing is an adhesion barrier to prevent the cells along the wall of the nasal cavity to adhere to each other during wound healing, thus fully or partially obstructing the lumen of the nasal cavity. In some embodiments, the barrier is an intranasal splint.

In some embodiments, the invention provides a dressing that can be placed in the nasal cavity by a healthcare provider (e.g., doctor or nurse) after sinus surgery to prevent adhesions, control mild bleeding, and provide a level of protection against bacteria and fungi. In some embodiments, the dressing is supplied as a compressed foam, and it dehydrates blood, thereby causing hemoconcentration of platelets, serum proteins and fibrinogen, leading to clotting that limits and controls bleeding and edema. In some embodiments, the dehydration of blood occurs quickly, and thus the hemoconcentration of platelets, serum proteins, and fibrinogen is rapid. In some embodiments, upon hydration, the dressing expands to contact and conform to the surrounding anatomy.

In some embodiments, the dressing is comprised of N, O-Carboxymethyl Chitosan derived from non-shell fish based chitosan, modified cellulose, and antimicrobial agents. In some embodiments, the dressing is fragmentable and eliminated from the site of application by natural excretion through the action of cilia.

As used herein, the term “dressing” means any type of substance or compound that can be placed adjacent to or over a wound in a patient (e.g., a human patient). Thus, the term dressing includes, without limitation, a film that can be painted onto the wound, a sponge that can be placed into adjacent or within a wound and that can compress and/or expand, a compound that can be placed such that is on or adjacent to a wound, or a film that can be wrapped around a wound (e.g., using an adhesive strip).

In some, embodiments, the dressing comprises at least one matrix and at least one agent, wherein the at least one agent and the at least one agent are water soluble.

In some embodiments, the dressing described herein comprises as matrix comprising a polymer having an ionic state and an agent having an ionic state, wherein the ionic state of the polymer is different from the ionic state of the agent. In some embodiments, the agent has a bactericidal activity, a fungicidal activity, and/or an antiviral activity. In some embodiments, the agent has an antimicrobial activity.

In some embodiments, the dressing described herein comprises a matrix comprising a polymer having an ionic state, where the polymer is anionic. In some embodiments, the polymer has a backbone that has a negative charge. In some embodiments, the polymer with a first ionic state is chitosan. Chitosan is a deacylated derivative of chitin and is soluble in aqueous acidic media via primary amine protonation. In some embodiments, chitosan is a linear polysaccharide composed of randomly distributed β-(1→4)-linked D-glucosamine (deacetylated unit) and N-acetyl-D-glucosamine (acetylated unit). In some embodiments, the chitosan is N,O-carboxymethyl chitosan (NOCC). In some embodiments, the N,O-carboxymethyl chitosan (NOCC) has the following structure:

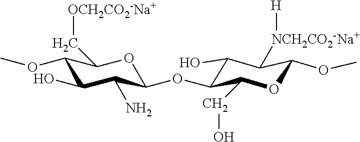

In some embodiments, the matrix of the dressing comprises a second polymer, said second polymer having a non-ionic state.

In some embodiments, the polymer having a non-ionic state is hydroxyethyl cellulose. In some embodiments, the structure of hydroxyethyl cellulose is:

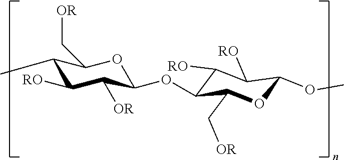

where n is be any integer greater than or equal to 1 and R is H or CH₂CH₂OH.

In some embodiments, the structure of hydroxyethyl cellulose is:

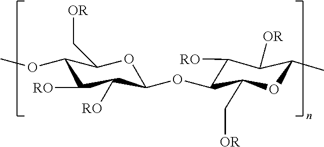

where n is be any integer greater than or equal to 1 and R is H, or CH₂CH₂OH, or CH₂CH₂OCH₂CH₂OH.

In some embodiments, the structure of hydroxyethyl cellulose is:

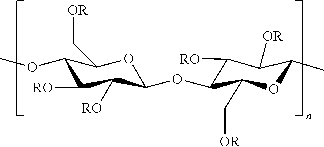

where n is be any integer greater than or equal to 1 and R is (CH₂CH₂O)_{y}H, where y is independently any integer.

In some embodiments, the structure for hydroxyethyl cellulose is:

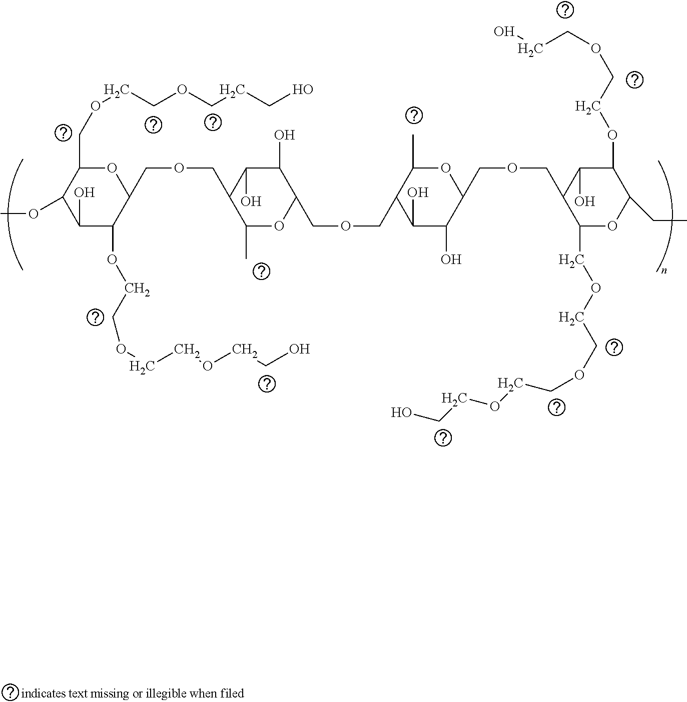

where n is be any integer greater than or equal to 1.

It will be understood that any number of compounds are employable in the matrix of the dressing described herein. For example, the following polymers, which are water soluble ionic polymers, may be included in the matrix of the dressing described herein: Anionic polymer salts coupled with a cation such as sodium (for example but other cations are possible), such as but not limited to sodium salt of polyacrylic acid, sodium salt of polyacrylamide, sodium salt of chitosan, such as a sodium salt of N,O-carboxymethyl chitosan, sodium salt of carboxymethyl cellulose, sodium alginate, sodium alginate sulfate, sodium salt of polymethacrylic acid, sodium salt of dextran sulfate, sodium salt of hyaluronic acid. Additional anionic polymers may be used. In another non-limiting example, the following water soluble cationic polymers may be used as the matrix in the dressing described herein: ammonium chloride salts of various polymers such as but not limited to poly (Diallyl Dimethyl Ammonium Chloride); ammonium bromide salts such as poly(2-vinyl-1-methylpyridinium bromide); hydrochloride salts such as poly (allylamine hydrochloride), chitosan hydrochloride.

It shall be understood that any combination of any polymer may be employed in the matrix of the dressing described herein.

In some embodiments, the matrix (and the dressing) is free of silver. In some embodiments, the matrix (and the dressing) is free of a silver salt. In other words, in some embodiments, the dressing described herein does not contain any silver. In some embodiments, the dressing herein does not contain any silver salt; for example, the dressing described herein does not contain the silver salt of a polymer.

In some embodiments, the dressing described herein further comprises an agent having a bactericidal activity, a fungicidal activity, an antiviral activity, a combination of a bactericidal and fungicidal activities, a combination of a fungicidal and antiviral activities, a combination of bactericidal and antiviral activities, and a combination of bactericidal, fungicidal activity, and antiviral activities. In some embodiments, when an agent or a combination of agents has bactericidal, fungicidal activity, and antiviral activities, that agent (or combination of agents) is said to be “anti-microbial”, or to have “anti-microbial activity.”

As used herein, by “bactericidal” is meant inhibiting the growth of a bacteria, including killing a bacteria. As used herein, by “fungicidal” is meant inhibiting the growth of a fungi, including killing a fungi. As used herein, by “antiviral” is mean inhibiting the growth of a virus, including killing a virus.

In some embodiments, the agent has an ionic state, where the ionic state of the agent is different than the ionic state of the matrix. For example, if the ionic state of the matrix is anionic, the ionic state of the agent is cationic. In some embodiments, the agent is cationic. In some embodiments, the agent has a positive charge center.

In some embodiments, the agent in the dressing is Methylene Blue. Methylene Blue has the following structure:

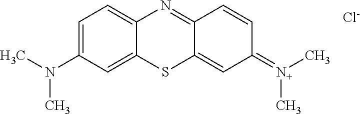

In some embodiments, the agent in the dressing is Gentian Violet (also sometimes called Crystal Violet). Gentian Violet has the following structure:

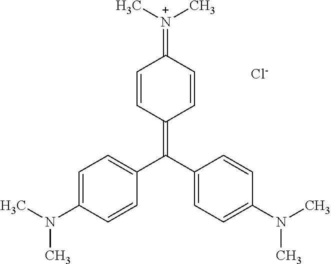

In some embodiments, both the methylene blue and gentian violet agents are in the dressing described herein. Both methylene blue and gentian violet are water soluble dyes that impart a blue color to water or other solution (e.g., physiological saline).

The combination of the Methylene Blue and Gentian Violet agents provides for a wide ranging anti-microbial efficacy against both gram negative and gram positive bacteria. It also is of note that the agents are antifungal as well.

It shall be understood that any number of agents may be used in the dressings described herein. For example, thionine dyes related to methylene blue that are electronegative or acidic may be used as agents. Additional agents include, without limitation, acridine orange, acridine yellow and related acriflavine (acridine) dyes which are electropositive or basic, quinacrine and its derivatives, brilliant green, bis naphthalene microbicides such as trypan blue and trypan, and triphenyl methane dyes that are related to crystal violet that are electropositive.

In some embodiments, the structural properties of the dressing described herein are enhanced as compared to identical formulation without the one or more agents. Without wishing to be bound by a particular theory, this enhanced structure in the dressing described herein may be due to the ionic state of the matrix (e.g., the NOCC polymer) and the opposite ionic state of the one or more agents (e.g., the methylene blue and/or gentian violet dyes), causing some ionic bonding between the one or more agents and the matrix that enhances the structure. The ionic bonding also controls the elution of the one or more agents (e.g., a dye) from the matrix of the dressing (e.g., a sponge)—and this ionic bonding allows controlled elution of the one or more agents from the dressing. In some embodiments, following the placement of the dressing in the nasal cavity, the elution of the one or more agents from the dressing is controlled by addition of elution liquid to the placed dressing by the health care provider (e.g., the surgeon) or the patient. When faster elution of the one or more agents from the dressing is desired, the ionic strength of the elution liquid is increased. When slower elution of the one or more agents from the dressing is desired, the ionic strength of the elution liquid is reduced. A non-limiting example of an elution liquid with high ionic strength is physiologically normal saline. For example, the elution liquid contains a physiologically normal amount of sodium chloride, which is about 0.9% w/v NaCl. A non-limiting example of an elution liquid with low ionic strength is deionized water. In some embodiments, the dressing has a shape with a length, width, and thickness and the one or more agents elute from the dressing into the wall of the lumen of the body cavity a distance of not more than 50% of the longest of said length, width, or thickness. It shall be understood that the shape may be any shape including, for example, a circle, rectangle, square, trapezoid, or a custom shape designed to fit into the desired body cavity. In some embodiments, the dressing has a length, width, and thickness and the one or more agents elutes from the dressing into the wall of the lumen of the body cavity a distance of not more than 25% of the longest of said length, width, or thickness. In some embodiments, the dressing has a length, width, and thickness and the one or more agents elutes from the dressing into the wall of the lumen of the body cavity a distance of not more than 10% of the longest of said length, width, or thickness.

In one nonlimiting example, if the dressing has a length of approximately 50 mm, a width of approximately 15 mm, and a thickness of 12 mm, the longest of the length, width, and thickness is the length, namely 50 mm. In some embodiments, where the one or more agents elutes from the dressing into the wall of the lumen of the body cavity a distance of not more than 50% of the longest of the length, width, or thickness, and the dressing is 50 mm long, 15 mm wide, and 12 mm thick, the one or more agents does not elute into the lumen of the body cavity more than 50% of 50 mm, or 25 mm. In another embodiment, where the one or more agents elutes from the dressing into the wall of the lumen of the body cavity a distance of not more than 10% of the longest of the length, width, or thickness, and the dressing is 50 mm long, 15 mm wide, and 12 mm thick, the one or more agents does not elute into the lumen of the body cavity more than 10% of 50 mm, or 5 mm.

In some embodiments, where the dressing comprises at least two agents, namely a first agent and a second agent, the first and second agent may elute from the dressing at different rates and may elute different distances into the wall of the lumen of a body cavity.

In some embodiments, the elution liquid (e.g., water) is added to the dressing externally (e.g., by the patient or a medical provider). For example, the patient with a nasal dressing may add elution liquid to his dressing by simply squirting elution liquid into his nasal cavity using, for example, an eye dropper or needleless syringe. In some embodiments, the bodily fluids released into the nasal cavity and the liquid providing moisture to the mucosal membrane in the nasal cavity have the same ionic strength as physiologically normal saline. In some embodiments, the bodily fluids released into the body cavity into which the dressing is placed acts as the elution liquid. For example, if the dressing is placed into a nasal cavity, bodily fluids of the patient (e.g., tears, nasal mucous, and phlegm) may act as the elution liquid, where contact of the dressing with the patient's bodily fluids results in elution of the antimicrobial agent from the dressing and, in some embodiments, the concomitant degradation of the dressing.

In some embodiments, both bodily fluids and externally added elution liquid act as elution liquids. In some embodiments, externally added elution liquid (e.g., water) may mix with a bodily fluid (e.g., nasal mucous) and this mixture is the elution liquid that is added to the dressing.

In some embodiments, the dressing further comprises a steroid. The addition of a steroid to the dressings described herein may lessen the amount of inflammation at the dressing placement area, thereby facilitating wound healing at the area. Any steroid may be used including, without limitation, triamcinolone acetonide, mometasone furoate, fluticasone proprionate, fluticasone furoate, budesonide, flunisolide, beclomethasone dipropionate, and corticosteroids such as cortisone, hydrocortisone, dexamethasone, and prednisone. In manufacturing the dressing described herein, in some embodiments, the steroid may be added during the manufacturing process. In some embodiments, the steroid may be added to the dressing at the time of use by the medical provider (e.g., physician or nurse). For example, when the physician placing the dressing in a body cavity of a patient is hydrating the compressed dressing with an expansion liquid (e.g., physiological saline), an amount of steroid may be added to the expansion liquid, thereby allowing the steroid to permeate through the dressing as it expands during hydration. In some embodiments, additional non-steroid medications can be added to the dressing either during manufacture or added by the medical provider during the placement of the dressing and subsequent hydration of said dressing by addition of the non-steroid medication to the expansion liquid.

The ordinarily skilled medical practitioner is able to determine the amount of steroid to add to the dressing considering factors including the size of the body cavity and dressing, the age of the patient, and the particular steroid being used. For example, an amount of steroid of between about 100 micrograms and about 5 grams may be added to the dressing. In one embodiment, an amount of steroid between about 100 micrograms and 1 gram may be added to the dressing. In some embodiments, an amount of steroid between about 500 micrograms and 2.5 grams may be added to the dressing. In some embodiments, between about 1 gram and 5 grams of steroid may be added to the dressing.

In some embodiments, where the steroid is added during the manufacturing step, the amount of steroid added to the dressing may be between about 0.001% weight of entire dressing and between about 10% weight of entire dressing prior to lyophilization. In some embodiments, the amount of steroid added to the dressing may be between about 0.01% weight of entire dressing and between about 7.5% weight of entire dressing prior to lyophilization. In some embodiments, the amount of steroid added to the dressing may be between about 0.1% weight of entire dressing and between about 5% weight of entire dressing prior to lyophilization. In some embodiments, the amount of steroid added to the dressing may be between about 0.5% weight of entire dressing and between about 5% weight of entire dressing prior to lyophilization. In some embodiments, the amount of steroid added to the dressing may be between about 1% weight of entire dressing and between about 2.5% weight of entire dressing prior to lyophilization.

Steroids, in general, tend to be lipophilic and thus non-ionic. Without wishing to be bound by a particular theory, in some embodiments, where the dressing described herein includes one or more steroids, the rate of elution of the one or more steroids from the dressing when said dressing is contacted with an elution liquid (which may be a bodily fluid of the patient, externally added by either the patient or a medical provider, or a mixture of both) may be different than the rate of elution of the one or more agents (e.g., an agent having antimicrobial activity) from the dressing.

In yet another aspect of the dressing described herein, where the steroid is added during the manufacturing step, the steroid may be added such that it can covalently bind (e.g., via a linker) to the ionic polymer in the matrix or, if present, to the non-ionic polymer in the matrix. In this manner, as the dressing degrades, the steroid is also released and elutes from the dressing. The rate of elution of the steroid from the dressing can thus be controlled by a number of factors including, without limitation, the degradation rate of the dressing, by the amount of steroid used, the specific steroid used, and the manner in which the steroid is covalently bound to the matrix.

The dressing described herein is degradable. As the elution liquid is added to the dressing, either by contact of elution liquid with the dressing externally via human intervention (e.g., the patient flushing his/her nose with water or saline), or by the body releasing moisture (e.g., in the form of saliva, mucous or tears), that contacts the dressing, each of the one or more agents in the dressing is released and elutes from the dressing. As the one or more agents in the dressing each has an ionic state that is different than that of the matrix, elution of the one or more agents from the dressing will break the ionic bond formed between the agent and the matrix, thereby reducing the structural integrity of the dressing. Over time, as the one or more agents continues to elute from the dressing, the dressing will degrade. As a result, there is no need to remove the dressing following full healing of the wound. Of course, however, a healthcare provider (e.g., a surgeon or nurse) can remove the dressing at any time including before full healing. The patient can also remove the dressing, or the dressing may be removed accidentally, for example, upon the patient sneezing or blowing his nose, once the dressing is shrunken in size due to degradation.

In some embodiments, the dressing serves as an adhesion barrier to cells aligning (or forming) the wall of the body cavity. This feature of the dressing prevents the cells in the healing wound from adhering to the dressing itself via some of the many adhesion molecules expressed by the cells aligning the wall of the body cavity (e.g., aligning the wall of the nasal cavity). While the dressing need not be removed because it is degradable, in the event of purposeful or accidental removal of the not fully degraded dressing, because cells do not adhere to the dressing, this removal will not re-injure the tissue by pulling away cells that have adhered to the dressing.

Additionally, during wound healing, it is common for cells to reach out to one another adhere to one another via various adhesion molecules including integrin family members (e.g., alpha3,beta1 and alpha6,beta 4), cadherins (e.g., E-cadherin), selectins (e.g., E-selectin or CD62), and other cell adhesion molecules (e.g., EpCAM or CD326). But in a body cavity such as a nasal cavity, wound healing that would obstruct or partially obstruct the cavity would reduce the ability of the human patient to breathe. Accordingly, it is important during wound healing to minimize the reduction in diameter of the lumen of the nasal cavity. In this manner, some embodiments, the dressing prevents cells in the wall of the lumen of the body cavity from growing into the lumen and reducing the diameter of the lumen.

In some embodiments, cells in the wall of the lumen of the body cavity will not adhere directly to the dressing. By “adhering directly” means that a cell surface adhesion molecule on a cell (e.g., an integrin or cadherin) binds directly to a molecule or atom in the dressing via a covalent or non-covalent bond.

In some embodiments, the dressing described herein adheres to ionic compounds in the mucous. In some embodiments, the matrix comprises a polymer that is mucosa-adhesive. As is well known, the mucosa (also known as the mucosal membrane or mucous membrane) contains positive charged groups. Without wishing to be bound by a particular theory, the polymer is the matrix may form ionic bonds with the positive charged groups in the mucosa. As a result, in some embodiments, the dressing described herein is mucosa-adhesive. In some embodiments, when the dressing in placed into the nasal cavity and expanded by the addition of an expansion liquid (e.g., water or saline), the mucosa-adhesive property of the dressing holds the barrier in position.

In various aspects, the invention provides a dry porous compound comprising a matrix with a first ionic state and an agent with a second ionic state, where the agent having an activity selected from the group consisting of a bactericidal activity, a fungicidal activity, and an antiviral activity, said first ionic state being different from said second ionic state, wherein hydration of the compound with an expansion liquid results in an expanded compound of a predetermined shape.

In some embodiments, the dressing described herein comprises the following amounts of matrix and agent:
Minimum total agent amount: 0.01 wt percent with 99.99 wt percent polymer matrix
Maximum total agent amount: 0.1 wt % with 99.9 wt % polymer matrix

Of course, in some embodiments, as discussed below, the maximum total agent amount is about 90 wt % agent with about 10 wt % matrix.

It will be understood that the type and amount of agent will depend upon what the dressing will be used for, and what pathogen is targeted. For example, if gram positive bacteria are being targeted, the agent may be gentian violet, although gentian violet is also bactericidal to some gram negative bacteria, as well as having antiviral and fungicidal activity. In another example, if gram negative bacteria are being targeted, the agent may be methylene blue, although methylene is also bactericidal to some gram positive bacteria, as well as having antiviral and fungicidal activity.

In some embodiments, both methylene blue and gentian violet are used as agents in the dressing described herein.

In some embodiments, the following ratios of methylene blue (MB) to gentian violet (GV) may be employed as agents in the dressing:
100 MB: 0 GV; 99:1; 90:10; 75:25; 70:30; 65:35; 60:40; 59:41; 58:42; 57:43; 56:44; 55:45; 54:46; 53:47; 52:48; 51:49; 50:50: 49:51; 48:52; 57:53; 46:54; 45:55; 44:56; 40:60; 43:57; 42:58; 41:59; 40:60; 35:65; 30:70; 25:75; 10:90; 1:99; 0 MB: 100 GV.

### Matrix:

For the matrix, when the ionic polymer is NOCC, the sodium salt of NOCC is used, so that NOCC is ionic.

Thus, for the matrix:
Minimal amount of NOCC is 0.1%
Maximal amount of NOCC is 100%

In some embodiments, a nonionic polymer is also used in the matrix. For example, nonionic hydroxyethyl cellulose is also used in, for example, the following amounts:

Minimal amount of hydroxyethyl cellulose is 0% Maximal amount of hydroxymethyl cellulose is 99.9%.

In some embodiments, the following ratios of an ionic polymer (e.g., NOCC) to a non-ionic polymer (e.g., hydroxyethyl cellulose) may be employed in the matrix, or in the dressing: 100 ionic: 0 nonionic; 99 ionic: 1 nonionic; 90:10 nonionic; 75 ionic: 25 nonionic; 70 ionic: 30 nonionic; 65 ionic: 35 nonionic; 60 ionic: 40 nonionic; 59 ionic: 41 nonionic; 58 ionic: 42 nonionic; 57 ionic: 43 nonionic; 56 ionic: 44 nonionic; 55 ionic: 45 nonionic; 54 ionic: 46 nonionic; 53 ionic: 47 nonionic; 52 ionic: 48 nonionic; 51 ionic: 49 nonionic; 50 ionic: 50 nonionic; 49 ionic: 51 nonionic; 48 ionic: 52 nonionic; 57 ionic: 53 nonionic; 46 ionic: 54 nonionic; 45 ionic: 55 nonionic; 44 ionic: 56 nonionic; 40 ionic: 60 nonionic; 43 ionic: 57 nonionic; 42 ionic: 58 nonionic; 41 ionic: 59 nonionic; 40 ionic: 60 nonionic; 35 ionic: 65 nonionic; 30 ionic: 70 nonionic; 25 ionic: 75 nonionic; 10 ionic: 90 nonionic; 1 ionic: 99 nonionic; 0.5 ionic: 99.5 nonionic; 0.1 ionic: 99.9 nonionic.

In some embodiments, the amount of agent to the amount of matrix in the dressing is variable. In some embodiments, by weight, the dressing may comprise the following ratios of agent weight percentage (single agent or combination of agents) to matrix weight percentage (single ionic polymer or combination of more than one polymer so long as one polymer is ionic):
0.01 wt % agent: 99.99 wt % matrix; 0.02 wt % agent: 99.98 wt % matrix; 0.05 wt % agent: 99.95 wt % matrix; 0.09 wt % agent: 99.91 wt % matrix; 0.1 wt % agent: 99.9 wt % matrix; 0.2 wt % agent: 99.8 wt % matrix; 0.3 wt % agent: 99.7 wt % matrix; 0.4 wt % agent: 99.6 wt % matrix; 0.5 wt % agent: 99.5 wt % matrix; 0.6 wt % agent: 99.4 wt % matrix; 0.7 wt % agent: 99.3 wt % matrix; 0.8 wt % agent: 99.2 wt % matrix; 0.9 wt % agent: 99.1 wt % matrix; 1 wt % agent: 99 wt % matrix; 2 wt % agent: 98 wt % matrix; 3 wt % agent: 97 wt % matrix; 4 wt % agent: 96 wt % matrix; 5 wt % agent: 95 wt % matrix; 6 wt % agent: 94 wt % matrix; 7 wt % agent: 93 wt % matrix; 6 wt % agent: 94 wt % matrix; 7 wt % agent: 93 wt % matrix; 8 wt % agent: 92 wt % matrix; 9 wt % agent: 91 wt % matrix; 10 wt % agent: 90 wt % matrix; 20 wt % agent: 80 wt % matrix; 30 wt % agent: 70 wt % matrix; 40 wt % agent: 60 wt % matrix; 50 wt % agent: 50 wt % matrix; 60 wt % agent: 40 wt % matrix; 70 wt % agent: 30 wt % matrix; 80 wt % agent: 20 wt % matrix; 90 wt % agent: 10 wt % matrix.

In some non-limiting examples, where the dressing comprises four components, namely an ionic matrix component, a non-ionic component, and two agents, specifically Gentian violet and methylene blue, prototype dressings can be made as follows:

Prototype A

| | Matrix | | Agent | |
|---|---|---|---|---|
| Chemical | NOCC | Hydroxyethyl | Gentian | Methylene |
| name | (sodium salt) | cellulose | violet | Blue |
| | | (nonionic) | | |
| | 50% total | 50% total | 50% total | 50% total |
| | weight matrix | weight matrix | weight agent | weight agent |

| | | |
|---|---|---|
| Total | Matrix is 99.99% total | Agent is 0.01% total |
| dressing | weight of dressing | weight of dressing |

Prototype B

| | Matrix | | Agent | |
|---|---|---|---|---|
| Chemical | NOCC | Hydroxyethyl | Gentian | Methylene |
| name | (sodium salt) | cellulose | violet | Blue |
| | | (nonionic) | | |
| | 60% total | 40% total | 40% total | 60% total |
| | weight matrix | weight matrix | weight agent | weight agent |

| | | |
|---|---|---|
| Total | Matrix is 99.90% total | Agent is 0.10% total |
| dressing | weight of dressing | weight of dressing |

Prototype C

| | Matrix | | Agent | |
|---|---|---|---|---|
| Chemical | NOCC | Hydroxyethyl | Gentian | Methylene |
| name | (sodium salt) | cellulose | violet | Blue |
| | | (nonionic) | | |
| | 46% total | 54% total | 44% total | 56% total |
| | weight matrix | weight matrix | weight agent | weight agent |

| | | |
|---|---|---|
| Total | Matrix is 99.92% total | Agent is 0.08% total |
| dressing | weight of dressing | weight of dressing |

Prototype D

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 43% total weight | 57% total weight | 40% total weight | 60% total weight |
| | matrix | matrix | agent | agent |

| | | |
|---|---|---|
| Total dressing | Matrix is 99.91% total | Agent is 0.09% total |
| | weight of dressing | weight of dressing |

Prototype E

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 43% total weight | 57% total weight | 40% total weight | 60% total weight |
| | matrix | matrix | agent | agent |

| | | |
|---|---|---|
| Total dressing | Matrix is 99.91% total | Agent is 0.09% total |
| | weight of dressing | weight of dressing |

Prototype F

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 47% total weight | 53% total weight | 40% total weight | 60% total weight |
| | matrix | matrix | agent | agent |

| | | |
|---|---|---|
| Total dressing | Matrix is 99.91% total | Agent is 0.09% total |
| | weight of dressing | weight of dressing |

Prototype G

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 45% total weight | 55% total weight | 60% total weight | 40% total weight |
| | matrix | matrix | agent | agent |

| | | |
|---|---|---|
| Total dressing | Matrix is 99.90% total | Agent is 0.10% total |
| | weight of dressing | weight of dressing |

Prototype H

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 46% total weight | 54% total weight | 40% total weight | 60% total weight |
| | matrix | matrix | agent | agent |

| | | |
|---|---|---|
| Total dressing | Matrix is 99.91% total | Agent is 0.09% total |
| | weight of dressing | weight of dressing |

Prototype I

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 55% total weight | 45% total weight | 55% total weight | 45% total weight |
| | matrix | matrix | agent | agent |

| | | |
|---|---|---|
| Total dressing | Matrix is 99.91% total | Agent is 0.09% total |
| | weight of dressing | weight of dressing |

Prototype J

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 46% total weight | 54% total weight | 60% total weight | 40% total weight |
| | matrix | matrix | agent | agent |

| | | |
|---|---|---|
| Total dressing | Matrix is 99.95% total | Agent is 0.05% total |
| | weight of dressing | weight of dressing |

Prototype K

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 43% total weight | 57% total weight | 60% total weight | 40% total weight |
| | matrix | matrix | agent | agent |

| | | |
|---|---|---|
| Total dressing | Matrix is 99.93% total | Agent is 0.07% total |
| | weight of dressing | weight of dressing |

Prototype L

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 56% total weight | 44% total weight | 45% total weight | 55% total weight |
| | matrix | matrix | agent | agent |

| | | |
|---|---|---|
| Total dressing | Matrix is 99.91% total | Agent is 0.09% total |
| | weight of dressing | weight of dressing |

Prototype M

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 100% total weight | 0% total weight | 40% total weight | 60% total weight |
| | matrix | matrix | agent | agent |

| | | |
|---|---|---|
| Total dressing | Matrix is 99.91% total | Agent is 0.09% total |
| | weight of dressing | weight of dressing |

Prototype N

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 43% total weight | 57% total weight | 0% total weight | 100% total weight |
| | matrix | matrix | agent | agent |

| | | |
|---|---|---|
| Total dressing | Matrix is 99.91% total | Agent is 0.09% total |
| | weight of dressing | weight of dressing |

Prototype O

| | Matrix | | Agent | |
|---|---|---|---|---|
| Chemical name | NOCC (sodium | Hydroxyethyl | Gentian violet | Methylene Blue |
| | salt) | cellulose | | |
| | | (nonionic) | | |
| | 47% total weight | 53% total weight | 100% total weight | 0% total weight |
| | matrix | matrix | agent | agent |

| | | |
|---|---|---|
| Total dressing | Matrix is 99.91% total | Agent is 0.09% total |
| | weight of dressing | weight of dressing |

It will be understood, of course, that an ionic matrix component other than NOCC (sodium salt) may be used in the dressing. In some embodiments, no nonionic matrix is present in the dressing. In some embodiments, where there is a nonionic matrix component present in the dressing, that component can be a nonionic matrix component other than hydroxyethyl cellulose. Additionally, while the above dressing prototypes use as an agent either gentian violet, methylene blue, or a combination of the two, other agents may be used including, without limitation, acridine orange, acridine yellow and related acriflavine (acridine) dyes, quinacrine and its derivatives, brilliant green, bis naphthalene microbicides such as trypan blue and trypan, and triphenyl methane dyes that are related to crystal violet.

In some embodiments, where the dressing comprises a steroid component, the steroid may be added to the dressings described herein. For example, 100 μg to 5 grams of any steroid (or a mixture of two or more steroids) may be used in the dressings described herein.

For example, a non-limiting steroid, namely triamcinolone acetonide, may be added to Prototype A dressing above to produce the following Prototype P

Prototype P

| | Matrix | | Agent | | Steroid |
|---|---|---|---|---|---|
| Chemical | NOCC | Hydroxyethyl | Gentian | Methylene | Triamcinolone |
| name | (sodium salt) | cellulose | violet | Blue | Acetonide |
| | | (nonionic) | | | |
| | 50% total | 50% total | 50% total | 50% total | 100% total |
| | weight matrix | weight matrix | weight agent | weight agent | weight steroid |

| | | | |
|---|---|---|---|
| Total | Matrix is 99.98% total | Agent is 0.01% total | Steroid is 0.01% total |
| dressing | weight of dressing | weight of dressing | weight of dressing |

In general, the dressing is manufactured by dissolving the polymers and dye agents in water (and, optionally, one or more steroid components) and then lyophilizing the solution to produce a dry porous sponge in a predetermined shape. To do this, the dressing in liquid form is placed into a mold in a predetermined shape. For use as a medical nasal dressing, the dry porous sponge is shaped into a predetermined shape that will fit into the nasal cavity of a human patient. During lyophilization, the dressing is frozen in place and then the water sublimates leaving behind the same general size and shape but now with air filling all the volume that was occupied by water. The sublimation process takes water in ice form and converts it to gaseous water vapor and bypasses the typical melting step. After lyophilization, the dry dressing is essentially the same size as the hydrated dressing.

In some embodiments, the dressing is next compressed into a flattened oval. This flattened oval then undergoes sterilization using gamma irradiation.

The dressing is used post surgery and placed in its compressed form by the physician next to or covering injured tissue to prevent adhesions by keeping the injured tissue separate from other tissue and also promotes healing by providing a moist environment for the wounded tissue. After placement, the physician will hydrate the compressed sterile sponge dressing with expansion liquid so that it will expand to its predetermined state. The agent(s) (e.g., antimicrobial, antibacterial, etc.) in the sponge dressing prevent the undesired growth of microbes to reduce the potential for infection. Agent that elutes into the wound would also locally reduce the potential for microbial growth. In use, the dressing is initially hydrated by the surgeon with an expansion liquid of water or saline, and then the patient is instructed to rehydrate the dressing frequently with an elution liquid over a period of time. Doing so allows the dressing to degrade and get naturally eliminated from the nasal passage.

### Example I

In this example, the dressing is manufactured by dissolving the N, O-Carboxymethyl Chitosan (NOCC) derived from non-shell fish based chitosan (sodium salt, therefore ionic), nonionic polymer hydroxyethyl cellulose, gentian violet, and methylene blue in water at the ratios described herein, and then lyophilizing the solution to produce a dry porous sponge in a predetermined shape. The dressing is then compressed and then sterilized.

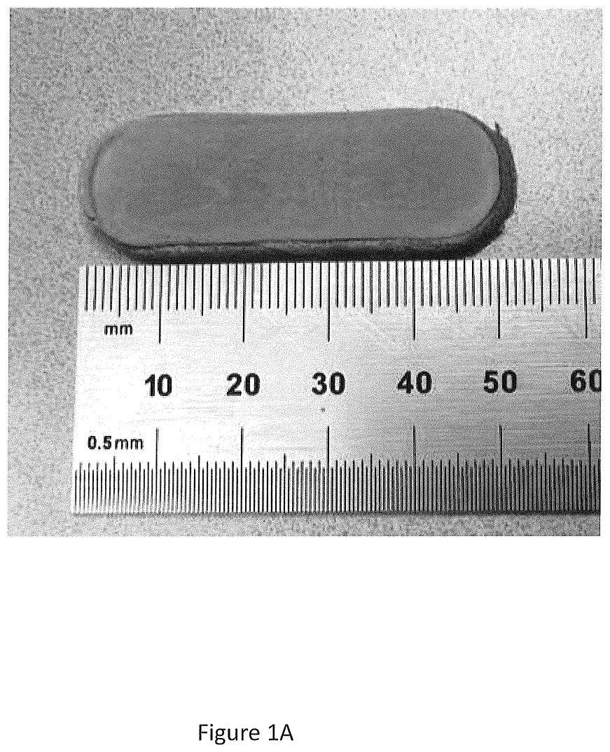

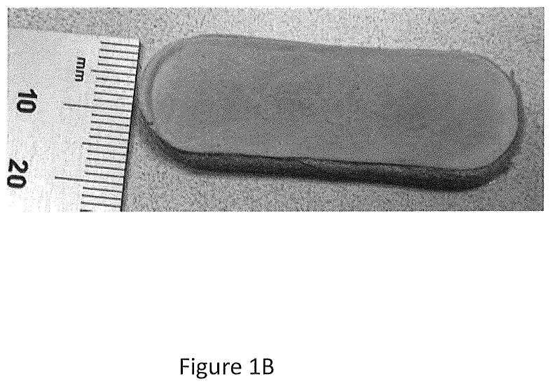

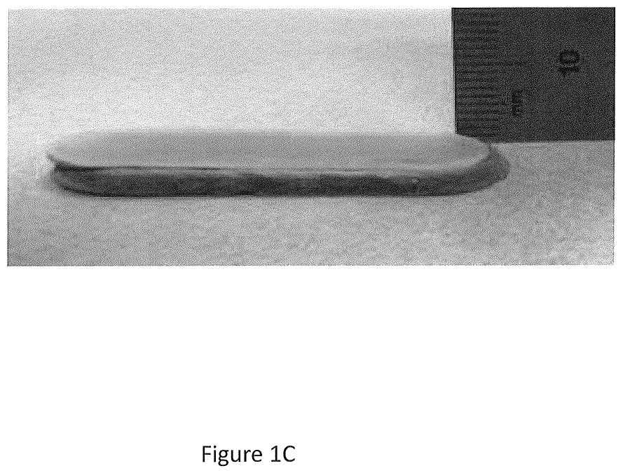

FIGS. 1A-1C are photographs of a compressed dressing shown from the top of the dressing (FIGS. 1A and 1B) and from the side (FIG. 1C). As can be seen, the dressing in this example, which is intended for placement into a nasal cavity, is approximately 50 mm in length, approximately 15 mm wide, and approximately 2 mm thick.

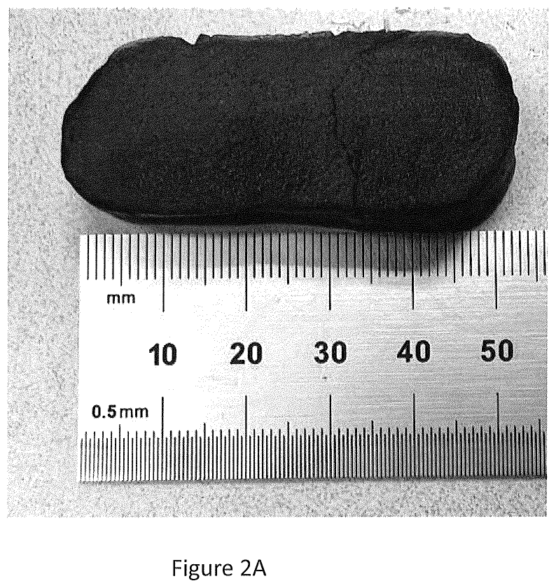

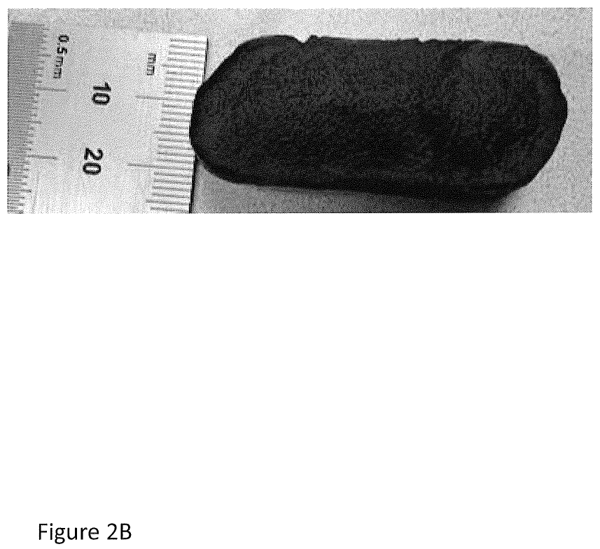

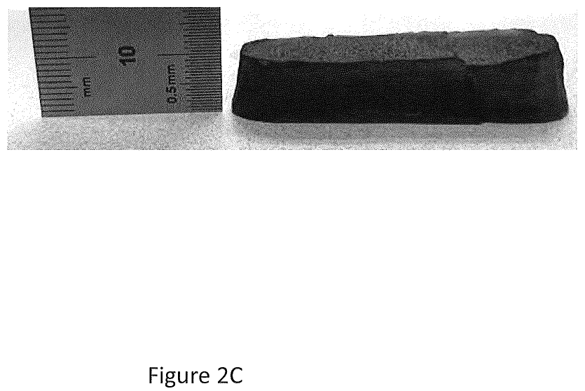

The sterile compressed dressing is hydrated with water, used as an expansion liquid in this example, to resume its predetermined shape. FIGS. 2A-2C are photographs of a hydrated dressing shown from the top of the dressing (FIGS. 2A and 2B) and from the side (FIG. 2C). As can be seen, the dressing in this example, which is intended for placement into a nasal cavity, is approximately 50 mm in length, approximately 15 mm wide—thus, these dimensions did not change upon compression and hydration. However, the thickness increased—in this example, the thickness is approximately 12 mm thick (FIG. 2C).

Elution of the one or more agents from the nasal dressing is based on the ionic strength of the elution liquid used for the elution. For example, deionized water used as an elution liquid has a very low ionic strength and, consequently, the agents (e.g., the dyes) will slowly elute from the dressing. However, with physiologically normal saline (e.g., 0.9% w/v NaCl) being used an elution liquid, the agents (e.g., the dyes) are effectively eluted from the dressing and locally into the surrounding tissue.

In the embodiments where the dressing also includes one or more steroids or other medication, the steroid (or other medication) elutes from the nasal dressing at a different rate than the one or more agents.

Microbial testing has shown a complete absence of viable cells after the sponge dressing was inoculated with various gram positive bacteria, gram negative bacteria and fungi (greater than 4-log or 99.9% reduction of all organisms tested) for up to 7 days. Therefore, the dressing described herein provides an extra degree of protection across a broad spectrum of microorganisms that are commonly associated with morbidities of the nose. The dressing quickly dehydrates blood, thereby causing rapid hemoconcentration of platelets, serum proteins and fibrinogen, leading to clotting that limits and controls bleeding and edema. The dressing is fragmentable and degradable. Nondegraded fragments of the dressing are easily eliminated from the site of application by natural excretion through the action of cilia with or without patient intervention (e.g., nose blowing or sneezing).

In a test of the antimicrobial ability of the dressing, the following log reductions for a combination of gram positive and gram negative bacteria, as well as fungi, that are known to exist in the nasal passages was achieved with the dressing:

| | 1 Day Log | 2 Day Log | 7 Day Log |
|---|---|---|---|
| Organism | Removal | Removal | Removal |
| Bacterial Strain (Gram Positive) | | | |
| Staphylococcus aureus | >5.04 | >5.67 | >6.48 |
| Methicillin Resistant | >6.48 | >6.48 | >6.48 |
| Staphylococcus aureus (MRSA) | | | |
| Streptococcus pneumoniae | >6.48 | >6.48 | >6.48 |
| Bacterial Strain (Gram Negative) | | | |
| Pseudomonas aeruginosa | >6.48 | >6.48 | >6.48 |
| Klebsiella pneumonia | >5.63 | >5.70 | >6.48 |
| Moraxella catarrhalis | >6.48 | >6.48 | >6.48 |
| Fungi Strain | | | |
| Candida albicans | >5.49 | >5.48 | >5.54 |
| Aspergillus brasiliensis | >4.63 | >4.54 | >5.11 |

### Example II

In this example, the dressing comprised of N, O-Carboxymethyl Chitosan derived from non-shell fish based Chitosan, modified Cellulose, and antimicrobial agents is placed in the nasal cavity after sinus surgery to prevent adhesions, control mild bleeding, and provide a level of protection against bacteria and fungi. The dressing is supplied as a compressed foam, and it quickly dehydrates blood, thereby causing rapid hemoconcentration of platelets, serum proteins and fibrinogen, leading to clotting that limits and controls bleeding and edema. Upon hydration, the dressing expands to contact and conform to the surrounding anatomy.

The dressing is fragmentable and eliminated from the site of application by natural excretion through the action of cilia.

### Example III

In this example, the intended use of the dressing is after nasal/sinus surgery as nasal dressings. Nasal Dressings are commonly used following endoscopic nasal/sinus surgery to prevent ongoing minimal bleeding and to facilitate the wound healing process. The dressing encompasses the indications for use in nasal/sinus surgery to control bleeding and provide tissue separation for proper healing.

### Biocompatibility:

Biocompatibility testing was performed using ISO 10993 Biological Evaluation of Medical devices and FDA guidance document “Use of International Standard ISO 10993-1, ‘Biological evaluation of medical devices-Part 1: Evaluation and testing within a risk management process”, issued Sep. 4, 2020. The dressing complies with the biocompatibility requirements for its intended use.

### Sterilization, Packaging, Shelf Life:

The dressing is sterilized using a validated gamma radiation method to assure a sterility assurance (SAL) of 10-6. Shelf life testing was performed on the sterile product according to the applicable standards and guidance documents.

### Performance Bench Testing:

Design verification testing was performed for the dressing to demonstrate physical and functional requirements were met. The dressing demonstrated greater than 4 log antimicrobial activity against a wide range of gram positive bacteria, gram negative bacteria, and fungal strains, including strains with known resistance to antibiotics: devices inoculated with bacteria and fungi demonstrated a complete absence of viable cells for up to 7 days.

### Antimicrobial Information:

The antimicrobial effectiveness of the dressing was tested against the following microbial strains and no viable cells were observed at any time point. The table below outlines the timeframe in which the dressing demonstrated antimicrobial activity to the tested microbial strains.

| | Organism No. from | | | |
|---|---|---|---|---|
| | the American Type | | | |
| Bacterial Strain | Culture Collection | | | |
| (Gram Positive) | (ATCC) | 24 hour | 48 hour | 7 days |
| Staphylococcus | 6538 | >4 log removal | | |
| aureus | | | | |
| Staphylococcus | 33591 | >4 log removal | | |
| aureus (MRSA) | | | | |
| Streptococcus | 6301 | >4 log removal | | |
| pneumoniae | | | | |

| | ATCC No. | 24 hour | 48 hour | 7 days |
|---|---|---|---|---|
| Bacterial Strain | | | | |
| (Gram Negative) | | | | |
| Pseudomonas | 9027 | >4 log removal | | |
| aeruginosa | | | | |
| Moraxella | 25240 | >4 log removal | | |
| catarrhalis | | | | |
| Klebsiella | 4352 | >4 log removal | | |
| pneumonia | | | | |
| Fungi Strain | | | | |
| Aspergillus | 16404 | >4 log removal | | |
| brasiliensis | | | | |

| | | |
|---|---|---|
| Candida albicans | 19231 | >4 log removal |

The embodiments of the invention described above are intended to be merely exemplary; numerous variations and modifications will be apparent to those skilled in the art. All such variations and modifications are intended to be within the scope of the present invention as defined in any appended claims.

1.-43. (canceled)

44. A dressing comprising a matrix with a first ionic state and an agent with a second ionic state, said agent having an activity selected from the group consisting of a bactericidal activity, a fungicidal activity, and an antiviral activity, wherein said first ionic state is different from said second ionic state.

45. The dressing of claim 44, wherein the first ionic state is an anionic state, and the second ionic state is a cationic state.

46. The dressing of claim 44, wherein the matrix comprises a chitosan, such as N, O-carboxymethyl chitosan (NOCC).

47. The dressing of claim 44, wherein the agent is selected from the group consisting of methyl blue and gentian violet.

48. The dressing of claim 44, wherein the dressing further comprises a second matrix with a nonionic state or a cationic state.

49. The dressing of claim 48, wherein the second matrix comprises hydroxyethyl cellulose.

50. The dressing of claim 44, wherein the agent enhances a structural property of the dressing as compared to the matrix alone.

51. The dressing of claim 44, wherein elution of the agent from the dressing degrades the dressing.

52. The dressing of claim 44, wherein the dressing is degraded when placed into a lumen of a body cavity adjacent to a wall of said lumen, wherein said cavity produces moisture or is able to receive moisture, said moisture acting as an elution liquid when in contact with the dressing.

53. The dressing of claim 52, wherein the body cavity is a nasal cavity.

54. The dressing of claim 52, wherein the dressing has a shape with a length, a width, and a thickness and the agent elutes from the dressing into the wall of the body cavity a distance of not more than 50% of the longest of said length, width, or thickness, or a distance of not more than 25% of the longest of said length, width, or thickness, or a distance of not more than 10% of the longest of said length, width, or thickness.

55. The dressing of claim 52, wherein cells in the wall of the lumen of the body cavity will not adhere directly to the dressing.

56. The dressing of claim 44, wherein the dressing consists essentially of a matrix consisting essentially of N, O-carboxymethyl chitosan (NOCC) and hydroxyethyl cellulose and an agent selected from the group consisting of gentian violet and methylene blue; or a matrix consisting essentially of N, O-carboxymethyl chitosan (NOCC) and an agent selected from the group consisting of gentian violet and methylene blue.

57. The dressing of claim 56, wherein the dressing consists essentially N, O-carboxymethyl chitosan (NOCC), hydroxyethyl cellulose, gentian violet, and methylene blue.

58. The dressing of claim 56, wherein the dressing consists essentially N, O-carboxymethyl chitosan (NOCC), gentian violet, and methylene blue.

59. The dressing of claim 44, wherein the dressing is free of a silver salt or is free of silver.

60. The dressing of claim 44, further comprising at least one steroid.

61. The dressing of claim 60, wherein the steroid is covalently bound to the matrix.

62. A dry porous compound comprising a matrix with a first ionic state and an agent with a second ionic state, said agent having an activity selected from the group consisting of a bactericidal activity, a fungicidal activity, and an antiviral activity, said first ionic state being different from said second ionic state, wherein upon hydration of the compound with an expansion liquid, said hydrated compound expands to form the dressing of claim 44.

63. The compound of claim 62, wherein the dressing has a predetermined shape.

64. The compound of claim 63, wherein the predetermined shape designed to fit into a nasal passage of a human.

65. The compound of claim 62, wherein the compound is a flat oval that, upon hydration, expands in one dimension or expands in two dimensions or expands in three dimensions.

66. The compound of claim 62, wherein said expansion upon hydration is between about four-fold and about ten-found in one dimension
